(19) United States
(12) Patent Application Publication
(10) Pub. No.: US 2012/0302998 A1
(43) Pub. Date: Nov. 29, 2012

(54) SKIN TREATMENT METHOD

(76) Inventor: Anne M. Sutorius, Las Vegas, NV (US)

(21) Appl. No.: 13/116,992

(22) Filed: May 26, 2011

(51) Int. Cl. A61M 1/00; B28D 1/00; A21C 15/04

(52) U.S. Cl. 604/540; 53/435; 125/2

(57) ABSTRACT

River rocks are used in a therapy regime whereby the river rocks are used to absorb excess oils from the skin of a user. In one method, river rocks are collected from a source (e.g., river or supplier), sorted for size and color, engraved with logo or other content, packaged and shipped to customers or retailers with instructions on use. Use comprises placement of the river rocks on oily portions of the user's skin for an appropriate time period allowing the river rock to absorb the excess oils. The river rocks are reusable after being washed with mild soap and water.

### FIELD OF THE INVENTION

The embodiments of the present invention relate to a method for treating skin. More particularly, a method of absorbing excess oils collecting on skin.

### BACKGROUND

Chronically oily skin will often be coarse, acne-ridden and prone to blackheads. Oily skin also results in many types of pimples and blemishes. Pores also become more enlarged as a result of excess oil production, which causes the pores to stretch and become larger. People with oily skin often have a chronic shine to their skin as a result of the oil constantly oozing out of their pores. Most people with oily skin have the most oil and shine on their foreheads, nose and chin.

Skin produces excess oil for various reasons including heredity, diet, hormonal imbalance, certain cosmetics and pregnancy. During adolescent years, skin increases the production of oil, which, as set forth above, results in blackheads, pimples and other types of acne. The body also produces excess oil when there is a hormonal imbalance, which can occur at any age. The excess oil often clogs pores, resulting in comedones that can become infected and turn into painful blemishes. Some people have oily skin due to heredity and must deal with controlling the excess oil production for the majority of their lives.

Thus, there exists a need for an effective oil removing method which does not negatively impact the skin and which is easy to administer. Advantageously, the method should not be damaging to the user or environment.

### SUMMARY

Accordingly, the embodiments of the present invention involve using river rocks in a therapy regime whereby the river rocks are used to absorb excess oils from the skin of a user. In one method, river rocks are collected from a source (e.g., river or supplier), sorted for size and color, engraved with logo or other content, packaged and shipped to customers or retailers with instructions on use. Use comprises placement of the river rocks on oily portions of the user's skin for an appropriate time period allowing the river rock to absorb the excess oils. The river rocks are reusable after being washed with mild soap and water.

Other variations, embodiments and features of the present invention will become evident from the following detailed description, drawings and claims.

### BRIEF DESCRIPTION OF THE DRAWINGS

FIG. 1 shows a cross-sectional view of human skin and associated anatomy;

FIG. 2 illustrates a series of river rocks of the type suitable for use with the embodiments of the present invention;

FIG. 3 illustrate a river rock engraved with content according to embodiments of the present invention;

FIG. 4 illustrates a river rock as packaged according to the embodiments of the present invention;

FIG. 5 illustrates a story packaged with the series of river rocks as shown in FIG. 4; and

FIG. 6 illustrates a flow chart detailing a method according to the embodiments of the present invention.

### DETAILED DESCRIPTION

For the purpose of promoting an understanding of the principles in accordance with the embodiments of the present invention, reference will now be made to the embodiments illustrated in the drawings and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would normally occur to one skilled in the relevant art and having possession of this disclosure, are to be considered within the scope of the invention claimed.

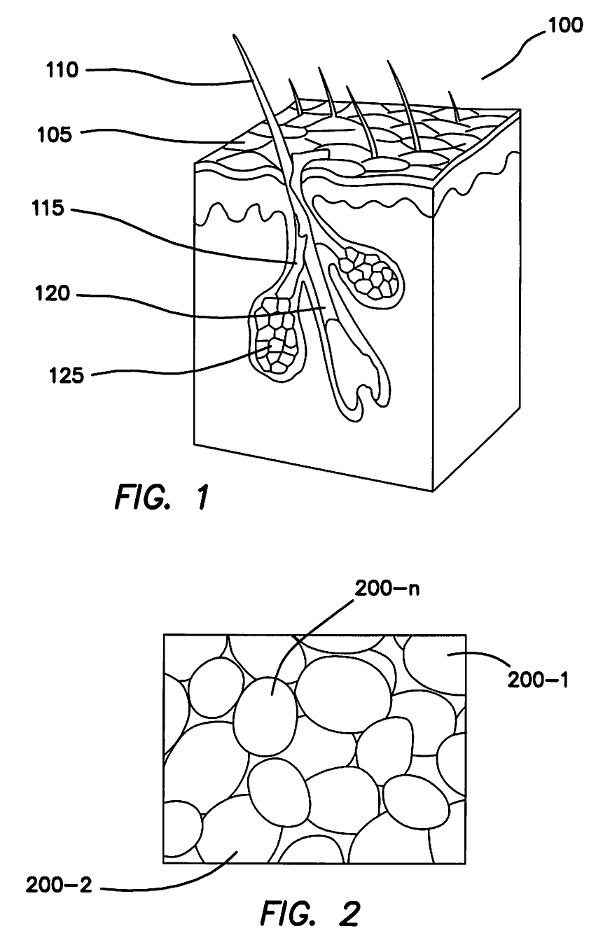

FIG. 1 shows a diagram 100 detailing a cross-sectional view of human skin and associated anatomy. The diagram shows a skin surface 105, hair 110, sebum 115, follicle 120, sebaceous gland 125. Acne is caused by an overproduction of sebum 115 (oil). The sebum 115 is formed by the sebaceous gland 125 under the skin surface 105. Sebum 115 normally flow along the follicle 120 to the skin surface 105 where the sebum 115 acts as a skin moisturizer. When follicles 120 become blocked by sebum 115, dead skin cells and bacteria, acne results. Acne usually forms on the face, neck and back.

Acne is the most common skin disease in the world. It affects 17 million people in the United States alone. Nearly 85% of people develop acne at some point between the ages of 12-25. While acne can not be cured, it can be treated. Current treatments include chemical-based medications and physician prescribed antibiotics. Benzoyl peroxide is the primary over-the-counter medication available for the treatment of acne. Medicated wipes have also generated traction in recent times. The basic two objectives of available treatments are to: (i) reducing bacteria; and (ii) drying excess oil. Unfortunately, none of the treatments has lasting effects and must therefore be used repeatedly. Moreover, many of the treatments have negative effects on the skin

FIG. 2 illustrates a series of river rocks 200-1 through 200-4 of the type suitable for use with the embodiments of the present invention. River rocks are stones having smooth rounded surfaces created by erosion caused by running water and weather conditions. For purposes of the embodiments of the present invention, river rocks have a diameter of 1″ to 2″ ideally. Smaller and larger river rocks are also acceptable for the embodiments of the present invention. Obtaining the river rocks is accomplished by either collecting them directly from river areas or via a supplier that has already collected them from river areas.

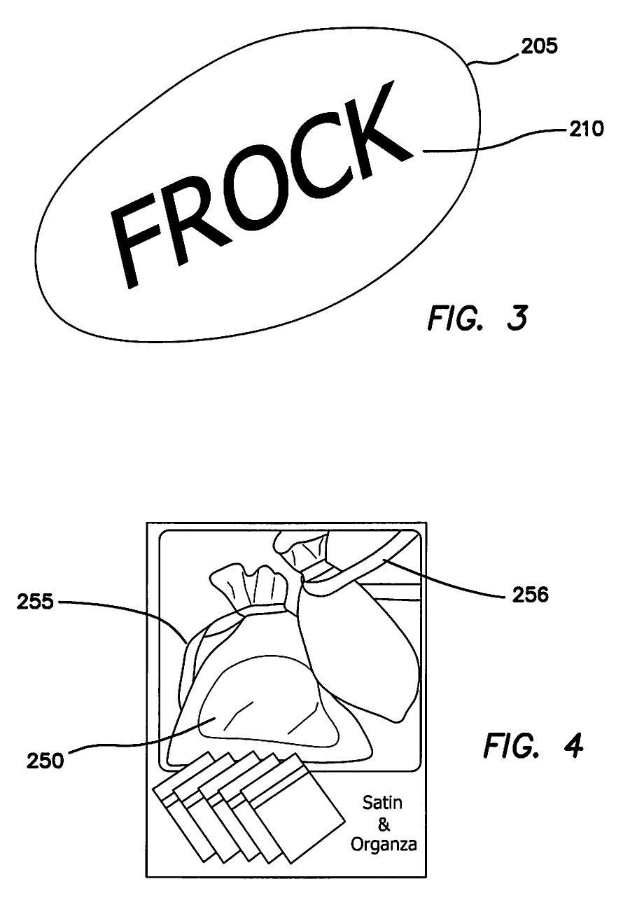

FIG. 3 shows a river rock 205 engraved with a logo 210. The engraving may be generated using sandblasting, lathing, carving, gouging or similar techniques. While a logo 210 is shown is FIG. 2, any content can be engraved into the river rocks. For example, as a gift, the river rocks can be engraved with a “Get Well” wish.

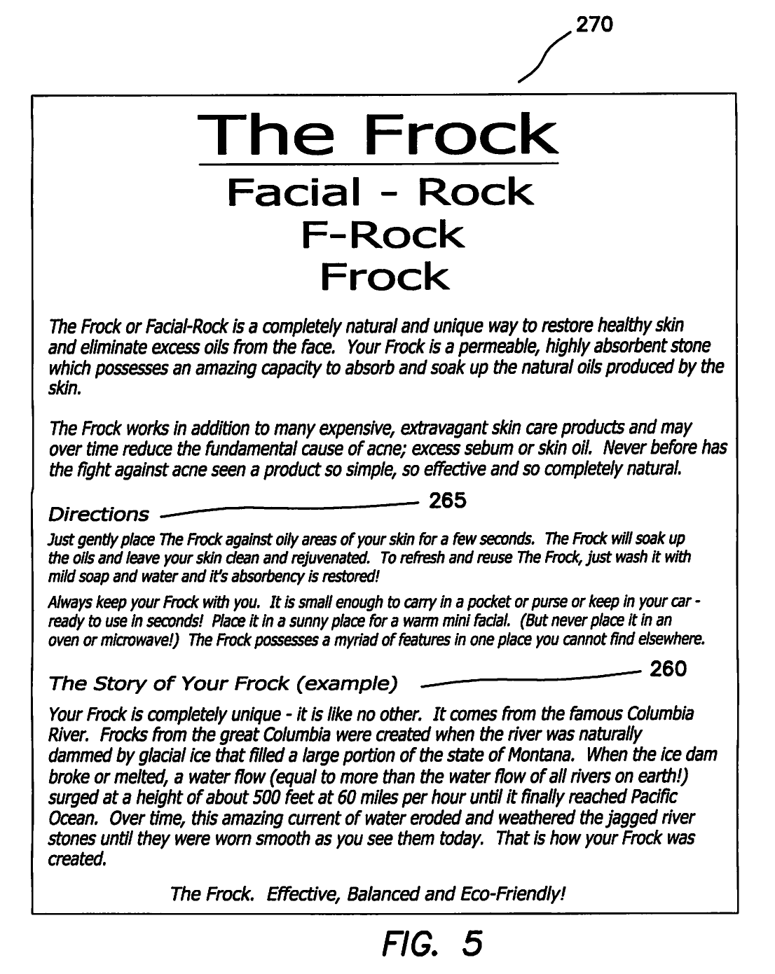

FIG. 4 shows a river rock 250 packaged in a satin (organza material, metallic lamé or other material), partially transparent pouch 255. The pouch 255 may be tied off with a string or ribbon 256 and have a rounded or flat bottom and is ideally sized 3″ by 4″ although any size is acceptable. Along with the river rock 250, the pouch 255 also retains a story 260 (see FIG. 5) of the river rock and directions 265 depicted on a parchment card 270. The story 260 explains the unique history of the retained river rock 250 lending a personal connection to the river rock 250 for the user.

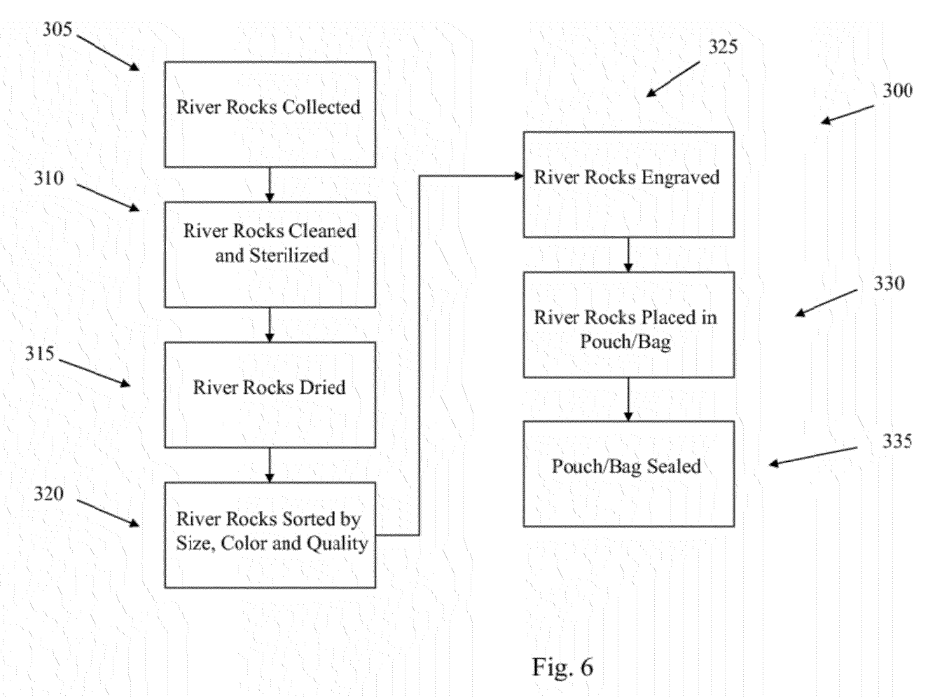

FIG. 6 shows a flow chart 300 detailing a method of collecting the river rocks. The steps of the flow chart 300 are not limited to the order presented and may thus be undertaken in a different order without departing from the spirit and scope of the embodiments of the present invention. At 305, the river rocks are either collected from river areas or suppliers having already done so. At 310, collected river rocks are cleaned and sterilized to remove dirt and bacteria. At 315, the river rocks are air dried. At 320, cleaned and sterilized river rocks are sorted by size, color and quality. At 325, the suitable river rocks are engraved with desired content. At 330, individual river rocks are placed in a pouch/bag with a parchment card. At 335, the pouch/bag is tied off retaining the river rock and parchment card therein.

Use of the river rock comprises placing the river rock on oily skin for a few seconds until the excess oil is absorbed. The river rock may be washed with a mild soap rendering it reusable. River rocks are effective, natural, portable, reusable, inexpensive and eco-friendly.

Although the invention has been described in detail with reference to several embodiments, additional variations and modifications exist within the scope and spirit of the invention.

I claim:

1. A method of treating skin comprising:
cleaning and sterilizing a river rock to remove dirt and bacteria; and
placing said river rock on oily skin for a pre-established time period sufficient for the river rock to absorb excess oil on the skin.

2. The method of claim 1 further comprising:
washing said river rock with a mild soap after use; and
re-placing the river rock on oily skin for a pre-established time period sufficient for the river rock to absorb excess oil on the skin.

3. A method comprising:
collecting river rocks;
cleaning and sterilizing said river rocks to remove dirt and bacteria;
drying said river rocks;
sorting by size, color and quality, said river rocks; and
engraving desired content on said sorted river rocks.

4. The method of claim 3 further comprising placing individual of said river rocks into a pouch.

5. The method of claim 4 further comprising placing a parchment card with directions and a river rock story in said pouch.

6. A method of treating skin comprising:
(a) cleaning and sterilizing a river rock to remove dirt and bacteria;
(b) placing said river rock on oily skin for a pre-established time period sufficient for the river rock to absorb excess oil on the skin;
(c) washing said river rock with a mild soap after use; and
(d) repeating steps (b) and (c) as needed.
